(19) United States
(12) Patent Application Publication
(10) Pub. No.: US 2014/0147530 A1
(43) Pub. Date: May 29, 2014

(54) TREATMENT PRODUCT WITH NATURAL CELLULAR DNA CARE INGREDIENT AND FUNCTIONAL COMPONENT

(71) Applicant: Benjamin Johnson, Evergreen, CO (US)

(72) Inventor: Benjamin Johnson, Evergreen, CO (US)

(21) Appl. No.: 14/090,950

(22) Filed: Nov. 26, 2013

Related U.S. Application Data
(60) Provisional application No. 61/729,911, filed on Nov. 26, 2012.

(51) Int. Cl. A61K 36/74; A61K 31/194; A61K 31/355; A61K 31/198; A61K 36/48; A61K 31/375

(52) U.S. Cl. CPC A61K 36/74; A61K 36/48; A61K 31/375; A61K 31/355; A61K 31/198; A61K 31/194
USPC 424/757; 424/725

(57) ABSTRACT

A treatment product is disclosed including a natural cellular DNA repair ingredient in combination with at least one other functional component. The natural cellular DNA repair ingredient may be derived from Uncaria tomentosa, Acacia plumose, and/or Uncana rhynchophylla. The treatment product may be internally administered, for example, where the at least one other functional component is selected from pyruvate, niacin, ubiquinone, lipoic acid, L-aspartic acid, citric acid, riboflavin, thiamine, potassium bicarbonate, sodium bicarbonate, and magnesium citrate. The treatment product may be topically administered, for example, where the at least one other functional component includes an amino acid, zinc and a hydroxylating active. The amino acid may be glycine, lysine, and/or proline. The hydroxylating active may be alpha-ketoglutarate and/or vitamin C. The at least one other functional component increases ATP production at a cellular level and/or enhances effectiveness of the natural cellular DNA repair ingredient at a cellular level.

### PRIORITY CLAIM

This application claims the benefit of U.S. Provisional Patent Application No. 61/729,911 filed on Nov. 26, 2012, titled “Treatment Product with Natural Cellular DNA Care Ingredient And Functional Component” of Benjamin Johnson, incorporated herein by reference for all that is disclosed as though fully set forth herein.

### BACKGROUND

Dermatologists typically distinguish skin conditions such as dry skin and the effect of aging, from a “disease” such as melanoma or skin cancer. There are a variety of products on the market, in addition to prescription formulations that can be used to treat skin conditions.

DNA repair is a process performed by the body over a trillion times everyday. Recent discoveries show that we can improve the amount and/or quality of DNA repair by assisting the body with certain active ingredients. DNA damage is believed to be a leading cause of aging and cell death.

### DETAILED DESCRIPTION

It has been postulated (and in many cases scientifically proven) that free radical damage is at the root of many diseases. It is believed that free radical damage is at the root of many diseases and aging itself. Free radicals from normal cellular activity, environmental pollutants and damaging chemicals including antibiotics, prescription medication and chemotherapeutic agents all lead to a more rapid decline in the health of cellular DNA.

Treatment products and methods of treating a skin condition with a natural cellular DNA repair ingredient in combination with at least one other functional component are disclosed. The natural cellular DNA repair ingredient is made from derivatives of medicinal plant species commonly known as “Cat's Claw” (e.g., plants such as Uncaria tomentosa and Acacia plumose and Uncana rhynchophylla).

In an example, the natural cellular DNA care ingredient is derived from the inner bark of Uncaria tomentosa, a vine indigenous to the Amazon rainforest. By way of illustration only, and not intended to be limiting, a commercially available example is AC-11® (by Optigenex, Inc. AC-11®). The natural cellular DNA care ingredient as implemented herein can be administered topically and/or orally, and goes beyond the effects realized by other antioxidant treatments to restore cell function. It is has been proven that this natural cellular DNA repair ingredient works with the body to repair DNA damage and boosts immune function. As such, the natural cellular DNA repair ingredient can be used to treat skin and other conditions, including those attributed to sun exposure, stress, and aging. It is noted, however, that the disclosure herein is not limited to the AC-11® formulation.

The treatment product disclosed herein may be formulated for internal administration and may include a natural cellular DNA repair ingredient (e.g., AC-11®) in combination with at least one other functional component, such as but not limited to at least one of the following: pyruvate, niacin, ubiquinone, and lipoic acid to name only a few examples. These functional components are selected to increase the natural production of ATP, which is a necessary part of DNA repair. ATP production is carefully regulated by the cells and is subject to bottlenecks in production in the Kreb's Cycle based on a lack of enzymes, nutrients and/or oxygen. ATP is needed for normal cellular function and DNA repair. By combining ingredients that increase ATP with ingredients like AC-11 that increase DNA repair, a significant increase in DNA repair along with improved health can be achieved.

The treatment product disclosed herein may also be formulated for external (e.g., topical) administration and may include a natural cellular DNA repair ingredient (e.g., AC-11®) in combination with amino acids and hydroxylating actives. The functional component may include, but is not limited to at least one amino acid (e.g., glycine, lysine, and proline), zinc and at least one hydroxylating active (e.g., alpha-keto glutarate, vitamin C). The skin has inherent mechanisms that assist in DNA repair. One of these systems utilizes zinc fingers. Topical components including but not limited to amino acids, zinc and vitamin C accelerate zinc finger formation thereby improving DNA repair. The combination of proven DNA repair ingredients like AC-11 along with zinc finger activation dramatically enhances the efficacy of either strategy creating younger, healthier skin.

In an example, a treatment product may be embodied as an internally administered supplement. In another example, a treatment product may be embodied as a topically administered agent. In either form, the treatment product may be used to treat a wide variety of skin conditions.

The internal supplement formula increases the production of ATP by augmenting the cell's natural ability to make ATP (Adenosine Triphosphate) through the aforementioned functional components. ATP is essential to good health and the more that is present in the cell the better every aspect of human health becomes. Cat's Claw is known to increase cellular components utilized in the manufacturing of ATP. The addition of one of the functional components described herein (including but not limited to pyruvate, coQ10, niacin, ubiquinone, lipoic acid, L-aspartic acid, citric acid, riboflavin, thiamine, potassium bicarbonate, sodium bicarbonate, and/or magnesium citrate) may further enhance ATP production. The combination of Cat's Claw and the functional components will also be beneficial in improving the healing capacity of the body. Research shows Cat's Claw can increase repair of human tissue. By providing the additional functional component(s), this formula enhances cellular repair as well.

An example of how this formulation benefits the body is improved athletic endurance.

Another example is improved mental clarity.

Another example is improved protein production throughout the body to include collagen, hormones, neurotransmitters, etc.

Another example is improved fat burning.

Another example is improved muscles growth and recovery.

Before continuing, it is noted that the term “skin condition” is defined herein according to well-accepted definitions known in the dermatology arts. For example, age spots are considered a skin condition. The mechanism of action of the treatment product is to repair damage to the dermal-epidermal junction, which reduces the signaling to the melanocyte that results in an age spot. Many treatments today reduce melanocyte production to lighten age spots, but the treatment product described herein goes to the source and heals the dermal-epidermal wound, thus preventing the age spot from forming in the first place. Accordingly the treatment product treats the origin of age spots. Of course, the treatment product may be used to treat other skin conditions, and is not limited to the repair of age spots.

It is noted that the treatment product includes the use of variations of a natural cellular DNA repair ingredient (e.g., AC-11®) in combination with at least one other functional component. That is, there are adaptations of the components that may be used to change its function for the purposes described herein.

In an example where the skin condition is hyperpigmentation, a build-up of DNA damage in the skin (specifically the melanocytes) results in the malfunction of melanocytes. The use of the treatment product increases the repair of DNA damage, in addition to increasing the repair of damaged proteins along the dermal-epidermal junction.

In an example where the skin condition is aging, an accumulation of DNA damage may reduce collagen production or healing capacity, Cat's Claw and zinc finger components will repair damaged cellular DNA and improve the recovery of aged skin.

The use of the topical treatment product may also be used for immune function, e.g., for the treatment of melasma, psoriasis, to slow and/or reverses akin aging effects.

### Example

Example treatment products have been formulated as described herein, as shown by the following test products. Each of the example treatment products included a natural cellular DNA repair ingredient in combination with at least one other functional component. The natural cellular DNA repair ingredient was derived from at least one of Uncaria tomentosa, Acacia plumose, and Uncana rhynchophylla.

When the product was internally administered, the at least one other functional component was selected from the group comprising at least one of pyruvate, niacin, ubiquinone, and lipoc acid.

When the product was topically administered the at least one other functional component was selected from the group comprising at least one of an amino acid, zinc and a hydroxylating active. The amino acid was selected from the group comprising at least one of glycine, lysine, and proline. The hydroxylating active was selected from the group comprising at least one of alpha-ketoglutarate and vitamin C.

Example Product 1: provided the most restoration for skin, and also included an aloe base for further hydrating. Example Product 2: provided enhanced DNA repair and collagen production with Vitamin C in a liquid base for quick penetration. Example Product 3: provided highly sensitive skin with improved strength, while treating all tested skin conditions. Sensitivity was also diminished during testing. Table I below shows example test cases:

TABLE 1

| | Product 1 | Product 2 | Product 3 |
|---|---|---|---|
| Skin Sensitivity | Normal to | Normal | Highly Sensitive |
| | Sensitive | | |
| Color | Deep Brown | Dark Brown, little | Deep Brown, |
| | (absorbs well) | color may settle | absorbs well |
| | | on skin | |
| Transport | Gel (Aloe) | Liquid | Gel (Aloe) |
| Agent/Texture | | | |

The example treatment products may be scented or unscented.

Testing showed that the functional component increased ATP production at a cellular level, and enhanced effectiveness of the natural cellular DNA repair ingredient at a cellular level.

In an example, the treatment products were clinically proven to increase DNA repair by at least 33% (over traditional methods), transforming skin repair by utilizing the powerful benefits of the formulation. The example treatment products each used a key component in the formation of collagen, alpha-ketoglutarate, with a lower dose L-Ascorbic acid, enabling more collagen and Zinc Finger Protein production, e.g., to benefit sensitive skin. The example treatment products further including amino acids resulted in visible healing, and measurable increases in collage and elastin production. The example treatment products further including Vitamin E enabled skin protection and repair by creating a super antioxidant when combined with Vitamin C to fight off free radicals.

In summary, the example treatment products resulted in more skin repair and rejuvenation through increased skin function, collagen production, and healing. Test subjects realized less hyperpigmentation, less UV damage, fewer fine lines and wrinkles, and improved wound repair (e.g., from acne).

It is noted that the examples shown and described are provided for purposes of illustration and are not intended to be limiting. Still other examples are also contemplated.

1. A treatment product including a natural cellular DNA repair ingredient in combination with at least one other functional component.

2. The treatment product of claim 1 wherein the natural cellular DNA repair ingredient is derived from Uncaria tomentosa.

3. The treatment product of claim 1 wherein the natural cellular DNA repair ingredient is derived from Acacia plumose.

4. The treatment product of claim 1 wherein the natural cellular DNA repair ingredient is derived from Uncana rhynchophylla.

5. The treatment product of claim 1 wherein the product is internally administered.

6. The treatment product of claim 5 wherein the at least one other functional component is selected from pyruvate, niacin, ubiquinone, lipoic acid, L-aspartic acid, citric acid, riboflavin, thiamine, potassium bicarbonate, sodium bicarbonate, and magnesium citrate.

7. The treatment product of claim 1 wherein the product is topically administered.

8. The treatment product of claim 7 wherein the at least one other functional component includes an amino acid, zinc and a hydroxylating active.

9. The treatment product of claim 8 wherein the amino acid is at least one of glycine, lysine, and proline.

10. The treatment product of claim 8 wherein the hydroxylating active is at least one of alpha-ketoglutarate and vitamin C.

11. The treatment product of claim 1 wherein the at least one other functional component increases ATP production at a cellular level.

12. The treatment product of claim 1 wherein the at least one other functional component enhances effectiveness of the natural cellular DNA repair ingredient at a cellular level.

13. A method of treating skin, comprising:
providing a natural cellular DNA repair ingredient in combination with at least one other functional component for administering to a treatment area.

14. The method of claim 13 wherein the natural cellular DNA repair ingredient is derived from at least one of Uncaria tomentosa, Acacia plumose, and Uncana rhynchophylla.

15. The method of claim 13 wherein the product is administered topically or internally.

16. The method of claim 13 wherein the at least one other functional component is selected from pyruvate, niacin, ubiquinone, lipoic acid, L-aspartic acid, citric acid, riboflavin, thiamine, potassium bicarbonate, sodium bicarbonate, and magnesium citrate.

17. The method of claim 13 wherein the at least one other functional component includes an amino acid, zinc and a hydroxylating active.

18. The method of claim 13 wherein the amino acid is at least one of glycine, lysine, and proline, and wherein the hydroxylating active is at least one of alpha-ketoglutarate and vitamin C.

19. The method of claim 13 further comprising increasing ATP production at a cellular level.

20. The method of claim 13 further comprising enhancing effectiveness of the natural cellular DNA repair ingredient at a cellular level.
